(19) United States
(12) Patent Application Publication
(10) Pub. No.: US 2022/0076791 A1
(43) Pub. Date: Mar. 10, 2022

(54) METHOD AND SYSTEM FOR FORMULATING A REQUIRED COMPOSITION FROM AT LEAST ONE INGREDIENT OF VARIABLE COMPOSITION

(71) Applicant: SIGMA ALIMENTOS, S.A. DE C.V., San Pedro Garza García, Nuevo León (MX)

(72) Inventors: Alfonso PEREZ GALLARDO, Apodaca, Nuevo León (MX); Nestor LUNA MARROQUIN, Villa de Santiago, Nuevo León (MX); Gregorio Jose DE HAENE ROSIQUE, Monterrey, Nuevo Leon (MX)

(21) Appl. No.: 17/416,087

(22) PCT Filed: Dec. 19, 2019
(86) PCT No.: PCT/MX2019/000141

§ 371 Date: Jun. 18, 2021

(30) Foreign Application Priority Data
Dec. 19, 2018 (MX) MX/A/2018/016129

(51) Int. Cl. G16C 20/30; A23C 9/12

(52) U.S. Cl. CPC G16C 20/30; A23C 2210/30; A23C 9/1223

(57) ABSTRACT

A method and system for formulating a required composition in order to produce a determined product from the supply of at least one ingredient that has a time-varying composition; the method has the steps of: (a) pre-establishing a final profile of components of the required composition; (b) providing a first ingredient and a second ingredient, where at least one of them has a time-varying composition; (c) determining an initial profile of components of the first ingredient and the second ingredient; (d) estimating an amount of the first ingredient and the second ingredient to be supplied; (e) supplying, simultaneously or sequentially, the estimated amount of the first ingredient and the second ingredient, and simultaneously, in-line and/or in real-time, generating a real-time profile of components of the amount of the first ingredient and the second ingredient as they are being supplied; (f) determining in-line or in real-time whether the amount of the first ingredient and/or the amount of the second ingredient has changed its composition when comparing the profiles; (g) adjusting the estimate of the amount of the first ingredient and/or of the second ingredient being supplied, if any of them has changed its composition; and repeating steps (e) to (g) until the required composition is achieved.

### TECHNICAL FIELD OF THE INVENTION

The present invention relates to methods and systems for formulating a composition. More specifically, the present invention relates to a method and system for formulating a suitable composition to make a determined product from ingredients whose composition varies over time.

### BACKGROUND OF THE INVENTION

At present, it is common to prepare compositions from the mixture or reaction of two or more ingredients of stable composition, in such a way that as the mixture or reaction is prepared, this is quantitatively and qualitatively analyzed in real-time in order to determine its composition, so that in a controlled and dosed way, amounts of one or another ingredient are added until as long as the mixture or reaction does not reach a certain objective composition, all within a determined volume or mass.

To carry out the above, in general, calibration curves are established for each of the constituents that must make up the target composition, these calibration curves are correlated in real-time, using partial least squares regression techniques, analysis of partial components, or multiple linear regression, with the composition of constituents of said mixture or reaction determined in a timely manner and in real-time by quantitative and qualitative spectral analysis techniques of optical transmission, optical reflectance, dispersion and fluorescence, such as mid-infrared spectroscopy (MIR) and near infrared spectroscopy (NIR). Examples of some embodiments of this type are described in U.S. Pat. Nos. 5,258,620, 6,639,044, 8,072,596 and 8,158,175; in US patent application publications US20100285186 and US20150306555; and in PCT international patent application publications WO0017611 and WO2015040626.

The techniques described in these patent documents have the disadvantage that they consider that the resulting compositions start from ingredients with a stable composition, so if a suitable composition is required to make a specific product from ingredients whose composition varies over time it cannot be to elaborate it with these techniques.

It is therefore necessary to offer a method and system for formulating in real-time a suitable composition to make a determined product from ingredients whose composition or constitution varies over time.

### SUMMARY OF THE INVENTION

In order to overcome the disadvantages of the state of the art described above, it is an object of the present invention to offer a method for formulating a composition required to produce a determined product, the method contemplates the steps of: (a) pre-establishing a final profile of components of the required composition to produce the determined product; (b) providing a first ingredient and a second ingredient, wherein at least one of these ingredients has a time-varying composition; (c) determining an initial profile of components of the first ingredient and an initial profile of components of the second ingredient; (d) estimating an amount of the first ingredient and an amount of the second ingredient to be supplied to form a composition close to the required composition, wherein these amounts are estimated by correlating the initial profile of components of the first ingredient and the initial profile of components of the second ingredient with the pre-established file profile of components; (e) supplying, simultaneously or sequentially, the estimated amount of the first ingredient and the estimated amount of the second ingredient, and simultaneously, in-line and/or in real-time, generating a real-time profile of components of the amount of the first ingredient as supplied and a real-time profile of components of the amount of the second ingredient as supplied; (f) determining in-line or in real-time whether at least one of the amounts of the first ingredient and the second ingredient has changed its composition, wherein said variation of composition is determined by simultaneously correlating the real-time profile of components of the amount of the first ingredient being supplied and the real-time profile of components of the amount of the second ingredient being supplied with the pre-established final profile of components; (g) adjusting the estimate of the amount of the first ingredient and/or the estimate of the second ingredient being supplied, under the determination that at least one of said amount of the first ingredient and amount of the second ingredient has varied its composition; and (h) repeating steps (e) to (g) until the close composition reaches the required composition to produce the determined product.

It is also an object of the present invention to offer a system for formulating a required composition in order to produce a determined product according to the method of claim 1, the system is formed by a first container to contain a first ingredient, the first container includes an outlet; a second container to contain a second ingredient, the second container includes an outlet; wherein at least one of said first ingredient and second ingredient has a time-varying composition; a first feeding line in communication with the outlet of the first container; a second feeding line in communication with the outlet of the second container; a first chemical composition sensor in the first feeding line; a second chemical composition sensor in the second feeding line; a storage tank in communication with the first feeding line and the second feeding line; a controller in communication with the first feeding line and the second feeding line; and a memory in communication with the controller for pre-establishing a final profile of components of a required composition to produce the determined product; wherein the first feeding line and the second feeding line are controlled by the controller to allow one or more amounts of the first ingredient and the second ingredient to be supplied to the storage tank; the first feeding line and the second feeding line are adapted to transmit mass or volume data of the amounts of the first ingredient and the second ingredient being supplied; the first chemical composition sensor and the second chemical composition sensor are adapted to transmit online and/or real-time data to the controller; and the controller is adapted to receive data from the first feeding line and the second feeding line, and from the first chemical composition sensor and the second chemical composition to estimate the amount of the first ingredient and the amount of the second ingredient to be supplied by the first feeding line and the second feeding line to the storage tank and to determine a component profile of the amounts of the first ingredient and the second ingredient and to correlate these profiles with the final profile of components pre-established in the memory.

### BRIEF DESCRIPTION OF THE DRAWINGS

The characteristic details of the present invention will be apparent from the following detailed description considered in connection with the accompanying drawings, for the purpose of defining the invention but without limiting its scope. It should be understood, however, that the drawings are made solely as an illustration and not as a limiting definition of the invention, in which:

FIG. 1 illustrates a system for formulating in real-time a fluid composition required to produce a determined product according to the present invention.

FIG. 2 illustrates a flow chart of a method for formulating in real-time a composition required to produce a determined product according to the present invention.

FIG. 3A illustrates a diagram of component concentration changes over time of a dairy ingredient as it empties from the tank (container) containing it, according to the present invention.

FIG. 3B illustrates a diagram of evolution over time of a concentration of components of a dairy food product composition during a mixing process according to the present invention.

FIG. 4A illustrates a diagram of feeding ingredients to a storage tank for preparing a whipped yogurt composition according to the present invention.

FIG. 4B illustrates a diagram of changes of composition of the whipped yogurt being prepared according to FIG. 4A in accordance with the present invention.

FIG. 5A illustrates a diagram of feeding ingredients to a storage tank for preparing a yogurt for drinking composition according to the present invention.

FIG. 5B illustrates a diagram of changes of composition of the yogurt for drinking being prepared according to FIG. 5A in accordance with the present invention.

FIG. 6A illustrates a diagram of feeding ingredients to a storage tank for preparing an indulgent yogurt composition according to the present invention.

FIG. 6B illustrates a diagram of changes of composition of the indulgent yogurt being prepared according to FIG. 6A in accordance with the present invention.

FIG. 7A illustrates a diagram of target values and real values measured according to the method of the present invention for various batches of preparation of whipped yogurt composition.

FIG. 7B illustrates a diagram of target values and real values measured according to the method of the present invention for various batches of preparation of yogurt for drinking composition.

FIG. 7C illustrates a diagram of target values and real values measured according to the method of the present invention for various batches of preparation of indulgent yogurt composition.

### DETAILED DESCRIPTION OF THE INVENTION

The characteristic details of this invention are described in the following paragraphs, which are intended to define the invention, but without limiting its scope.

Under the context of the present description, the term “time-varying composition” means a composition that over time undergoes some phenomenon that alters its composition, chemical concentration and/or state of aggregation during industrial stages of storage, processing and/or transport; some examples of these phenomena are: synthesis, decomposition, addition, aggregation, substitution, stratification, decantation, precipitation, sedimentation, chemical reactions, oxidation, combustion, gas evolution, heat absorption, heat evolution, acidification, alkalization, evaporation, coagulation, flocculation, curdling, distillation, condensation, protein interactions, caramelization, enolization, isomeration, dehydration, thermal degradation, enzymatic modifications, gelatinization, retrogradation, viscosity changes, crystallization, covalent bonding, derivatization, hydrolysis, putrefaction, proteolysis, resynthesis, transacylation, deamidation, desulfurization, lipolysis, retrogradation.

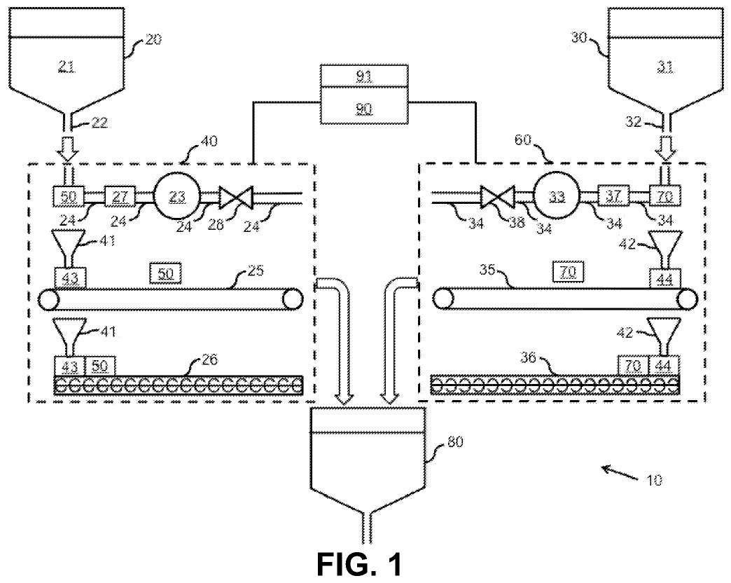

FIG. 1 illustrates a system 10 for formulating in real-time a composition required to produce a determined product according to the present invention. The system 10 includes at least a first container 20, a second container 30, a first feeding line 40, a first chemical composition sensor 50, a second feeding line 60, a second chemical composition sensor 70, a storage tank 80 and a controller 90.

The first container 20 and the second container 30 store a first ingredient 21 and a second ingredient 31, respectively. The first ingredient and/or the second ingredient has a time-varying composition. The first ingredient 21 and the second ingredient 31 can be liquids, solids, powders and combinations thereof.

The first container 20 includes an outlet 22 in communication with the first feeding line 40, while the second container 30 includes an outlet 32 in communication with the second feeding line 60. The first feeding line 40 and the second feeding line 60 in turn are in communication with the storage tank 80. In an alternative embodiment, the first feeding line 40 and the second feeding line 60 may be in communication with a mixing manifold (not shown) and this in turn in communication with storage tank 80.

The first ingredient 21 and the second ingredient 31, contained in the first container 20 and second container 30, respectively, are supplied to the storage tank 80 through the first feeding line 40 and the second feeding line 60, respectively.

In a first embodiment, when the first ingredient 21 and the second ingredient 31 are liquids, the first feeding line 40 and the second feeding line 60 may be made up of pumps 23 and 33, respectively, arranged in or between conduits 24 and 34, respectively. In a second and third embodiment, when the first ingredient 21 and the second ingredient 31 are solids or powders, the first feeding line 40 and/or the second feeding line 60 may be made up of conveyor belts 25 and 35, respectively, or auger feeders 26 and 36, respectively. In view of this, it is apparent to a person skilled in the art to carry out any combination of these embodiments of configurations of the first feeding line 40 and the second feeding line 60, according to the type of first ingredient 21 and second ingredient 31 used in the present invention.

When using a system of pumps 23 and 33 as a configuration of the first feeding line 40 and/or the second feeding line 60, in order to ensure adequate amounts of the first ingredient 21 and the second ingredient 31 in liquid state to be pumped to storage tank 80 or mixing manifold (not shown) use is made of meters 27 and 37 and valves 28 and 38 which may be upstream or downstream of pumps 23 and 33, respectively. The meters 27 and 37 preferably measure mass or volume so that a precise amount of first ingredient 21 and/or second ingredient 31 flows into storage tank 80 or mixing manifold (not shown). The meters 27 and 37 can measure, respectively, the volume of the first ingredient 21 and the second ingredient 31 that are pumped, but can also measure other properties, such as mass or flow rate. The valves 28 and 38 can be of any type and can include a regulator that controls the rate and/or pressure of the flow of first ingredient 21 and/or second ingredient 31. The valves 28 and 38 control flow rates and/or flow. pressure of the first ingredient 21 and/or of the second ingredient 31 to ensure that the velocities and/or pressures of these at the moment of being fed to the storage tank 80 or to the mixing manifold (not shown) correspond to an adequate flow.

At the outlet 22 of the first container 20 and at the outlet 32 of the second container 30 or in or between the respective conduits 24 and 34 are located the first chemical composition sensor 50 and the second chemical composition sensor 70, respectively, however, in an alternative embodiment, the first chemical composition sensor 50 and the second chemical composition sensor 70 may be before or after the pumps 23 and 33, respectively. The first chemical composition sensor 50 and the second chemical composition sensor 70 make it possible to determine, in real-time, a component profile of the first ingredient 21 and the second ingredient 31.

The pumps 23 and 33 and the valves 28 and 38 are activated and controlled by the controller 90 based on the amounts of the first ingredient 21 and second ingredient 31 that in real-time are determined, according to the method of the present invention, to be fed to storage tank 80 or mixing manifold (not shown). The meters 27 and 37 transmit to the controller 90 data corresponding to the mass or volume of the amounts of the first ingredient 21 and the second ingredient 31 being fed to the storage tank 80 or to the mixing manifold (not shown), while the first chemical composition sensor 50 and the second chemical composition sensor 70 transmit data to the controller 90 to determine the profile of components of the first ingredient 21 and the second ingredient 31, respectively, and then these data are used by the controller 90 to control the pumps 23 and 33, and valves 28 and 38 throughout system 10 to ensure that precise chemical compositions and concentrations of the amounts determined and fed of the first ingredient 21 and the second ingredient 31 are used to mix and form a composition required to produce a determined product. These data transmissions from meters 27 and 37 and from the first chemical composition sensor 50 and the second chemical composition sensor 70 to the controller 90 and the activation and control of the pumps 23 and 33, and the valves 28 and 38 by of the controller 90 are carried out through the use of electrical and/or electronic or radio frequency signals, so that there is electrical, electronic, optical or electromagnetic communication between these components of the system 10.

When using a conveyor system 25 and 35 or auger feeder 26 and 36 as the configuration of the first feeding line 40 and/or the second feeding line 60, in order to ensure adequate amounts of the first ingredient 21 and the second ingredient 31 in solid or powder state to be transported to the storage tank 80 or to the mixing manifold (not shown), use is made of dosing hoppers 41 and 42 in communication with the outlet 22 of the first container 20 and at the outlet 32 of the second container 30, respectively and of mass meters 43 and 44 located in outlets of the dosing hoppers 41 and 42. The mass meters 43 and 44 can be scales or load sensors and measure, respectively, the amount of mass of the first ingredient 21 and second ingredient 31 being dosed. The first chemical composition sensor 50 and the second chemical composition sensor 70 are located on the conveyor belts 25 and 35 or at the entrance of the auger feeders 26 and 36. The first chemical composition sensor 50 and the second chemical composition sensor 70 allow determining, in real-time, a profile of components of the first ingredient 21 and the second ingredient 31, respectively.

The speed of advance of the conveyors belts 25 and 35 or of the auger feeders 26 and 36 and the opening and closing of the dosing hoppers 41 and 42 are controlled by the controller 90 based on the amounts of the first ingredient 21 and second ingredient 31 that are determined in real-time, according to the method of the present invention, to be transported to the storage tank 80 or the mixing manifold (not shown). The mass meters 43 and 44 transmit to the controller 90 data corresponding to the mass of the amounts of the first ingredient 21 and the second ingredient 31 being transported to the storage tank 80 or to the mixing manifold (not shown), while the first chemical composition sensor 50 and the second chemical composition sensor 70 transmit data to controller 90 to determine the profile of components of the first ingredient 21 and the second ingredient 31, respectively, and then these data are used by the controller 90 to control the conveyor belts 25 and 35 or auger feeders 26 and 36 and dosing hoppers 41 and 42 throughout the system 10 to ensure precise chemical compositions and concentrations of the amounts determined and fed of the first ingredient 21 and the second ingredient 31 are used to mix and form a composition required to produce a determined product. These data transmissions from the mass meters 43 and 44 and the first chemical composition sensor 50 and the second chemical composition sensor 70 towards the controller 90 and the activation and control of the conveyor belts 25 and 35 or the auger feeders 26 and 36 and the dosing hoppers 41 and 42 by the controller 90 are made by using electrical and/or electronic signals, so there is electrical and/or electronic communication between these components of the system 10.

The first chemical composition sensor 50 and the second chemical composition sensor 70 can be an electrochemical transducer, optical transducer, mass transducer, thermal transducer, acoustic transducer, and combinations thereof; among the optical transducers are those of optical transmission, optical reflectance, dispersion, fluorescence and their combinations, either near infrared spectroscopy, visible light spectroscopy, fluorescence spectroscopy, Raman spectroscopy, surface-enhanced Raman spectroscopy (SERS), spectroscopy of other wavelengths within the magnetic spectrum, magnetic resonance and combinations thereof. Some examples of chemical composition sensors available in the state of the art and applicable to the present invention are listed in Table 1.

TABLE 1

| Denomination or | | Method or functional | Manufacturing |
|---|---|---|---|
| trade name | Analysis parameter | principle | company |
| Measurement of | Humidity | Microwave humidity | PCE Instruments/Aquar System |
| humidity in the | | sensor | Ltd |
| process | | | |
| Source | Humidity | Resonance | Source |
| Technology | | Hot oven | technology |
| | Protein and fat | NIR | |
| Online | Humidity y solids | Ultrasound LS-SVM | Unidentified |
| measurement of | | | |
| particle | | | |
| concentrations | | | |
| Milktronics | Turbidity | Optical | Milktronics |
| | Viscosity | Online viscometer | |
| | Solids | Optical/osmosis | |
| PendoTECH | Protein | Continuous flow UV | PendoTECH |
| | | sensor | |
| BioPAT ®Spectro | Multiparameter | Flexible set of | Sartorius STEDIM |
| | | detectors for UV/VIS | |
| | | and NIR spectra | |
| NIR QUEST | Multiparameter | Configurable NIR/ | Ocean OPTICS |
| | | Spectrometer with | |
| | | configurable combs | |
| Milkostream | Single point | FTIR | FOSS |
| | multiparameter for low | | |
| | viscosity fluids only | | |
| Milk-Inspector | Multiparameter | NIR | Quality2Process |
| | | | B.V. |
| | | | J&M Analytik AG |
| Unidentified | Lactose | Enzyme sensor - | Lund University |
| | | Cellobiose | |
| | | dehydrogenase based | |
| | | sensor | |
| Opti-I Relative | Solids and Turbidity | NIR | Metrom |
| Turbidity Sensor | | | instruments |
| THz | Hz spectroscopy has | Terahertz | Unidentified |
| | potential for the | spectroscopy | |
| | quantitative analysis of | | |
| | milk fat, total solid, | | |
| | lactose, milk protein, | | |
| | casein, and somatic | | |
| | cells. | | |
| Conductivity | Salts, solids, cryoscopic | Conductivity | Unidentified |
| | point (indirect) | | |
| Bio-transducers | Salts, proteins, | Impedance | Unidentified |
| (Nanosensors, | carbohydrates, toxins, | Amperometry | |
| biosensors) | contaminants, | Potentiometry | |
| | pathogens or viruses | Conductometry | |
| | | Optical | |
| | | Raman spectroscopy | |
| | | SERS spectroscopy | |

The controller 90 can be any programmable device that is pre-programmed, programmable by an operator, programmed and reprogrammed by a closed-loop logic system or preferably all of these are available in a single controller 90. The controller 90 has an integrated memory 91 and may have different user interfaces, such as keyboards, displays, touchscreens, switches, audible sound generators, and others.

The controller 90 is preferably a Central Processing Unit (“CPU”) or another programmable device such as a Printed Circuit Board (“PCB”) to control, according to the configuration adopted of the first feeding line 40 and/or second feeding line 60, pumps 23 and 33, valves 28 and 38, meters 27 and 37, conveyor belts 25 and 35, auger feeders 26 and 36, dosing hoppers 41 and 42, as well as receiving the information coming from meters 27 and 37, mass meters 43 and 44, the first chemical composition sensor 50 and the second chemical composition sensor 70. A composition required to produce a determined product is preferably programmed into the controller 90 in order to control the amounts, feed rates, conveying rates and pressures with which the first ingredient 21 and/or second ingredient 31 must be fed to the storage tank 80 or the mixing manifold ado (not shown).

The system 10, in particular the first feeding line 40 and the second feeding line 60, can include a set of instruments to acquire process variables such as probes, spectrometers, transducers, thermocouples, pressure meters, density meters, flow meters, pH meters, among others (not shown).

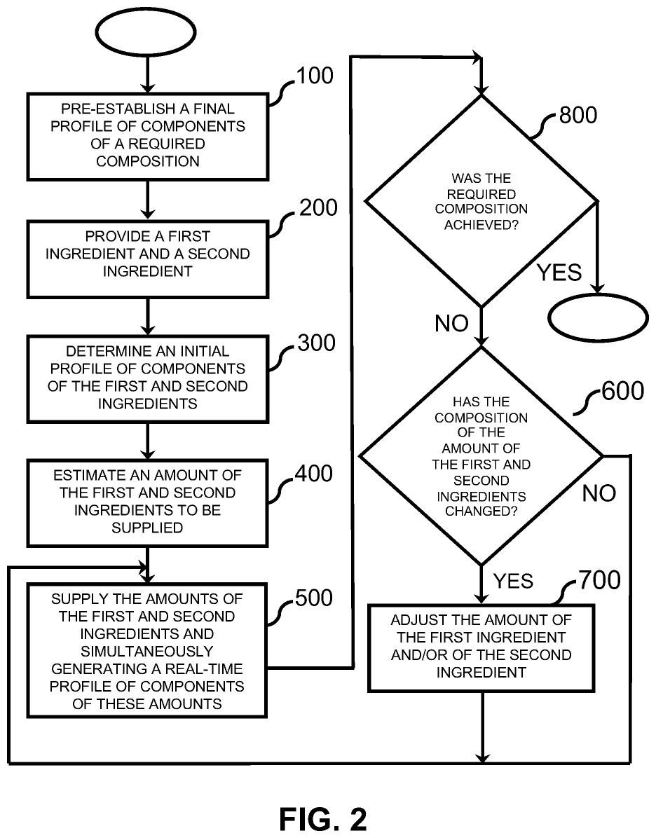

Now with reference to FIG. 2, a method for formulating in real-time a composition required to produce a determined product according to the present invention is described below in relation to the system 10 of FIG. 1.

The method can start in step 100, where a final component profile of a composition required to produce a determined product is pre-established in memory 91 of controller 90, this final component profile is generally determined and established by the using calibration curves for each of the constituents that must make up the composition required to produce a determined product, these calibration curves can be prepared as described in U.S. Pat. No. 5,258,620, the content of which is incorporated by reference.

At step 200, first container 20 and second container 30 provide a first ingredient 21 and a second ingredient 31, respectively, such that either or both of the first ingredient 21 and second ingredient 31 have a time-varying composition, due to any of the phenomena mentioned above.

Then in step 300 an initial profile of components of the first ingredient 21 and an initial profile of components of the second ingredient 31 are determined in the controller 90, by means of a transduction that the first chemical composition sensor 50 performs to the first ingredient 21 and that the second chemical composition sensor 70 performs to the second ingredient 31, the transduction information from the first chemical composition sensor 50 and the second chemical composition sensor 70 are transmitted to the controller 90 for processing in order to correlate it and thus determine the initial profile of components of the first ingredient 21 and the initial component profile of the second ingredient 31.

Once the initial component profile of the first ingredient 21 and the initial component profile of the second ingredient 31 have been determined, the same controller 90, in step 400, estimates, by means of a mass balance, an amount of the first ingredient 21 and an amount of the second ingredient 31 to be supplied to form a composition close to the composition required to produce the determined product, these amounts are estimated by correlating the initial component profile of the first ingredient 21 and the initial component profile of the second ingredient 31 with the final profile of components pre-established in memory 91.

Then, in step 500, the controller 90 orders to the first feeding line 40 and the second feeding line 60 to supply, simultaneously or sequentially, the amount of the first ingredient 21 and the amount of the second ingredient 31 estimated, and simultaneously, online and/or in real-time, generate a real-time profile of components of the amount of the first ingredient 21 as it is supplied and a real-time profile of components of the amount of the second ingredient 31 as it is supplied, this through a online and/or real-time transduction that the first chemical composition sensor 50 performs on the amount of the first ingredient 21 as it is being supplied and that the second chemical composition sensor 70 performs on the amount of the second ingredient 31 as it is supplied, the transduction information from the first chemical composition sensor 50 and the second chemical composition sensor 70 is transmitted online and/or in real-time to the controller 90 for its processing in order to determine online or in real-time, in step 600, if one or both of said amount of the first ingredient 21 and amount of the second ingredient 31 has varied its composition by simultaneously correlating the real-time profile of components of the amount of the first ingredient 21 and the real-time profile of components of the amount of the second ingredient 31 with final profile of components pre-established in step 100.

In case that the controller 90 determines that either or both of the amount of the first ingredient 21 and the amount of the second ingredient 31 has varied its composition, then the controller 90, in step 700, adjusts the estimate of the amount of the first ingredient 21 and/or the amount of the second ingredient 31 being supplied, ordering to the first feeding line 40 and/or the second feeding line 60 to supply the amount of the first ingredient 21 and/or the amount of the second ingredient 31 which have been suitable, and as long as the close composition does not reach the required composition to produce the determined product, step 800, proceed with the repetition of steps 500, 600, 700 and 800.

### EXAMPLES OF EMBODIMENT OF THE INVENTION

The invention will now be described with respect to the following examples, which are solely for the purpose of representing the manner of carrying out the implementation of the principles of the invention. The following examples are not intended to be an exhaustive representation of the invention, nor are they intended to limit the scope of the invention.

The examples that are illustrated below are applications of the principles described above of the present invention to prepare compositions of dairy food products, however it will be evident for a person skilled in the art to apply said principles of the invention in the preparation of any other composition in any technical field of application.

### Example 1

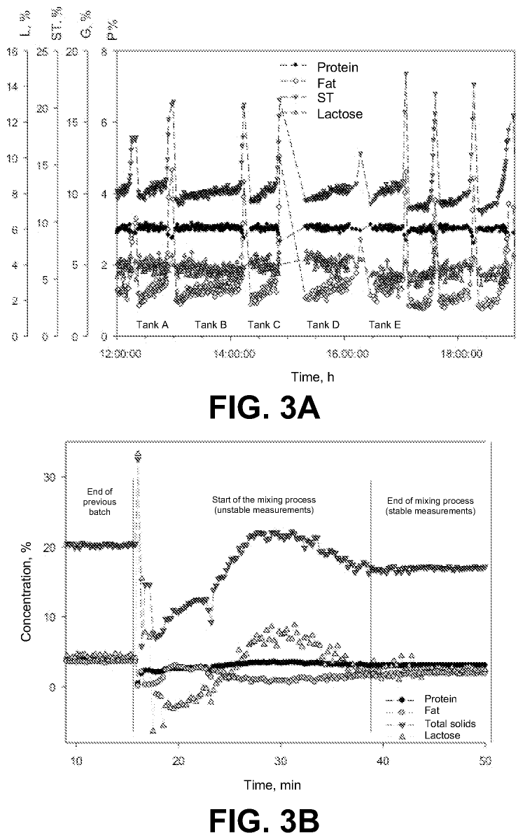

In relation to FIG. 3A, it shows a diagram of a stratification phenomenon that a dairy ingredient undergoes as it is emptied from the tank (container) that contains in order to prepare a composition required for a dairy food product, thus observing the changes in the concentration of each of the components of the dairy ingredient over time, where “G” is fat, “L” is lactose, “P” is protein, and “ST” is total solids; while FIG. 3B shows a diagram of evolution over time of the concentration of components of the composition required for a dairy food product being prepared by applying the method and system of the present invention.

### Example 2

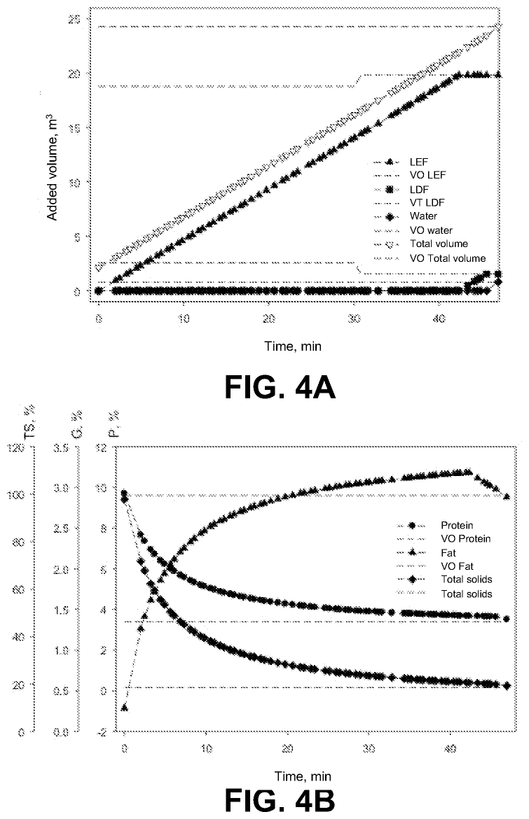

A composition of whipped yogurt was prepared, using as ingredients water, fluid whole milk (LEF) and fluid skim milk (LDF) that were fed according to the method and system of the invention to the storage tank as shown in the diagram of the FIG. 4A, wherein it is also observed the target value of whole skimmed milk (VO LEF), the total volume of fluid skim milk (VT LDF), target value of water (VO water), total volume and the target value of the total volume (VO total volume); while the composition changes of whipped yogurt during the filling of the storage tank is shown in the diagram of FIG. 4B, wherein the components shown are protein (P), fat (G) and total solids (TS) with respect to the target value of protein (VO protein), target value of fat (fat VO) and total solids, respectively. The desired target values (VO) and the real values (measured by methods of the present invention) are shown in FIGS. 4A and 4B, with very tight control of the required composition.

### Example 3

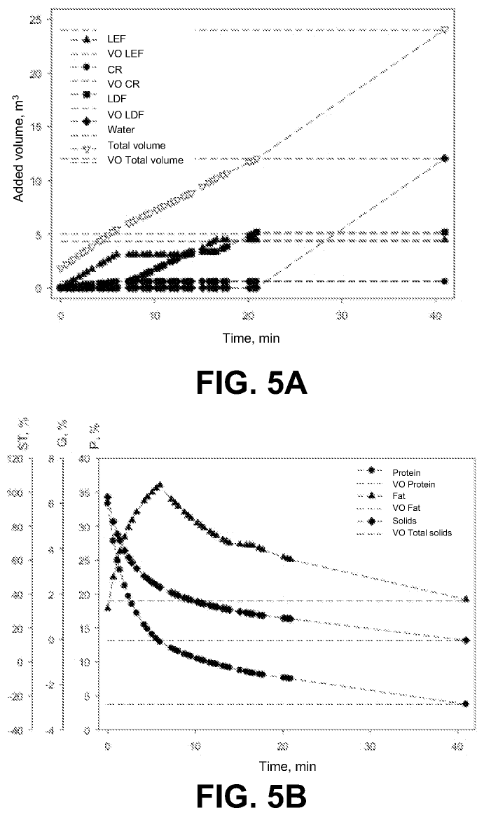

A composition of yogurt for drinking was prepared, using as ingredients water, fluid whole milk (LEF), fluid skim milk (LDF) and cream (CR) that were fed according to the method and system of the invention to the storage tank according to shown in the diagram of FIG. 5A, wherein it is also observed the target value of whole skimmed milk (VO LEF), the total volume of fluid skim milk (VT LDF), target value of water (VO water), target value of cream (VO CR), total volume and the target value of total volume (VO total volume); while the composition changes of yogurt for drinking during the filling of the storage tank is shown in the diagram of FIG. 5B, wherein the components shown are protein (P), fat (G) and total solids (TS) with respect to the target value of protein (VO protein), target value of fat (fat VO) and target value of total solids (total solid VO), respectively. The desired target values (VO) and the real values (measured by methods of the present invention) are shown in FIGS. 5A and 5B, with very close control of the required composition.

### Example 4

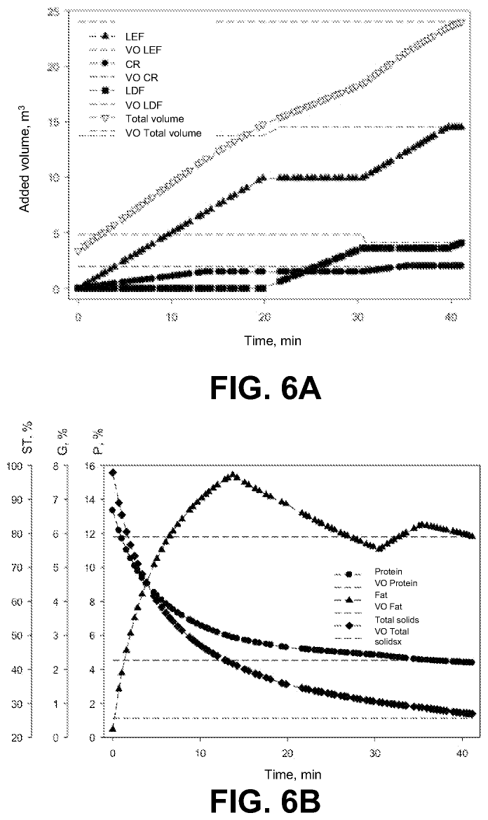

An indulgent yogurt composition was prepared, using as ingredients fluid whole milk (LEF), fluid skim milk (LDF) and cream (CR) that were fed according to the method and system of the invention to the storage tank as shown in the diagram of FIG. 6A, wherein it is also observed the target value of whole skimmed milk (VO LEF), the total volume of fluid skim milk (VT LDF), target value of cream (VO CR), total volume and the target value of the total volume (VO total volume); while the composition changes of indulgent yogurt during the filling of the storage tank is shown in the diagram of FIG. 6B, wherein the components shown are protein (P), fat (G) and total solids (TS) with respect to the target value of protein (VO protein), target value of fat (fat VO) and target value of total solids (total VO solids), respectively. The desired target values (VO) and the real values (measured by methods of the present invention) are shown in FIGS. 6A and 6B, with very close control of the required composition.

As seen in Examples 3, 4 and 5, after the mass balance calculation, the method and system of the present invention supplies the volume of liquid ingredients to the storage tank. During the filling of the storage tank to formulate the required composition, the method and system of the present invention controls the correct amount of each ingredient that must be supplied through the feeding lines, every time it is recalculated (adjusted) in real-time the amount of each ingredient being supplied when a change in the composition of any of these is detected.

### Example 5

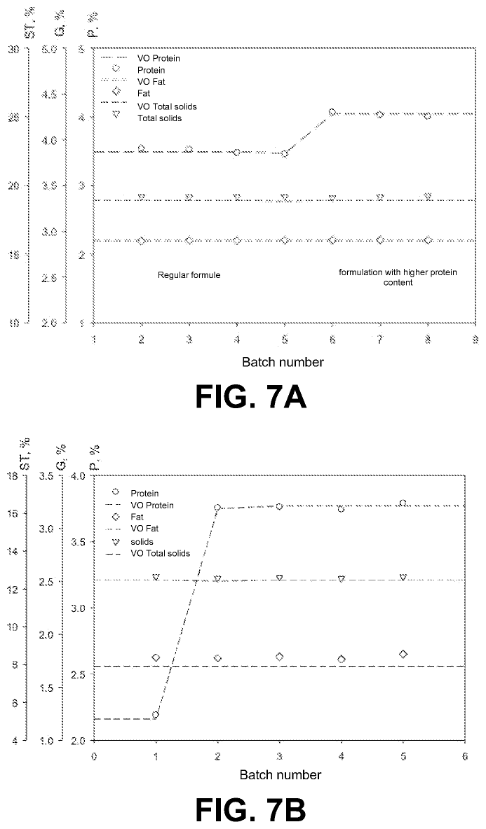

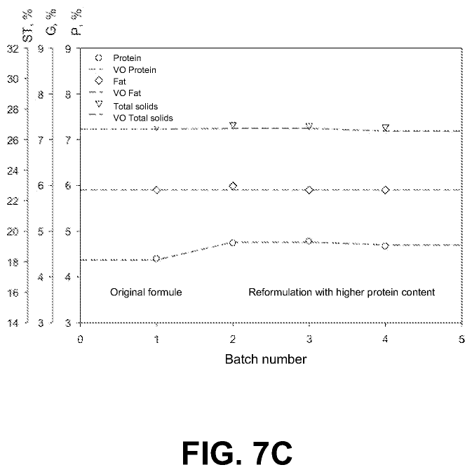

FIGS. 7A, 7B and 7C illustrate diagram of target values and real values measured according to the method of the present invention for various batches of composition preparation of whipped yogurt, yogurt for drinking and indulgent yogurt, respectively, wherein the components shown are protein the (P), fat (G) and total solids (TS) with respect to the target value of protein (VO protein), target value of fat (VO fat) and target value of total solids (VO total solids), respectively, for 5 batches of compositions, so it is observed that the method and system of the present invention allow a very close control of the composition.

Based on the embodiments described above, it is contemplated that modifications to the described embodiments, as well as alternative embodiments, will be apparent to a person skilled in the art under the present disclosure. It is therefore contemplated that the claims encompass such modifications and alternatives that are within the scope of the present invention or its equivalents.

1. A method for formulating a required composition in order to produce a determined product, the method comprises of the steps of:
(a) pre-establishing a final profile of components of the required composition to produce the determined product;
(b) providing a first ingredient and a second ingredient, wherein at least one of these ingredients has a time-varying composition;
(c) determining an initial profile of components of the first ingredient and an initial profile of components of the second ingredient;
(d) estimating an amount of the first ingredient and an amount of the second ingredient to be supplied to form a composition close to the required composition, wherein these amounts are estimated by correlating the initial profile of components of the first ingredient and the initial profile of components of the second ingredient with the pre-established file profile of components;
(e) supplying, simultaneously or sequentially, the estimated amount of the first ingredient and the estimated amount of the second ingredient, and simultaneously, in-line and/or in real-time, generating a real-time profile of components of the amount of the first ingredient as supplied and a real-time profile of components of the amount of the second ingredient as supplied;
(f) determining in-line or in real-time whether at least one of the amounts of the first ingredient and the second ingredient has changed its composition, wherein said variation of composition is determined by simultaneously correlating the real-time profile of components of the amount of the first ingredient being supplied and the real-time profile of components of the amount of the second ingredient being supplied with the pre-established final profile of components;
(g) adjusting the estimate of the amount of the first ingredient and/or the estimate of the second ingredient being supplied, under the determination that at least one of said amount of the first ingredient and amount of the second ingredient has varied its composition; and
(h) repeating steps (e) to (g) until the close composition reaches the required composition to produce the determined product.

2. The method of claim 1, wherein the first ingredient and the second ingredient are selected from a group consisting of liquid ingredients, solid ingredients, powered ingredients and combinations thereof.

3. The method of claim 1, wherein the initial profile of components and the real-time profile of components are determined by transduction selected from the group consisting of electrochemical transduction, optical transduction, mass transduction, thermal transduction, acoustic transducer and combinations thereof.

4. The method of claim 3, wherein the optical transduction is selected from the group consisting of optical transmission, optical reflection, dispersion, fluorescence, and combinations thereof.

5. A system for formulating a required composition in order to produce a determined product according to the method of claim 1, the system comprises:
a first container to contain a first ingredient, the first container includes an outlet;
a second container to contain a second ingredient, the second container includes an outlet;
wherein at least one of said first ingredient and second ingredient has a time-varying composition;
a first feeding line in communication with the outlet of the first container;
a second feeding line in communication with the outlet of the second container;
a first chemical composition sensor in the first feeding line;
a second chemical composition sensor in the second feeding line;
a storage tank in communication with the first feeding line and the second feeding line;
a controller in communication with the first feeding line and the second feeding line; and
a memory in communication with the controller for pre-establishing a final profile of components of a required composition to produce the determined product;
wherein the first feeding line and the second feeding line are controlled by the controller to allow one or more amounts of the first ingredient and the second ingredient to be supplied to the storage tank; the first feeding line and the second feeding line are adapted to transmit mass or volume data of the amounts of the first ingredient and the second ingredient being supplied; the first chemical composition sensor and the second chemical composition sensor are adapted to transmit online and/or real-time data to the controller; and the controller is adapted to receive data from the first feeding line and the second feeding line, and from the first chemical composition sensor and the second chemical composition to estimate the amount of the first ingredient and the amount of the second ingredient to be supplied by the first feeding line and the second feeding line to the storage tank and to determine a component profile of the amounts of the first ingredient and the second ingredient and to correlate these profiles with the final profile of components pre-established in the memory.

6. The system of claim 5, wherein the first ingredient and the second ingredient are selected from a group consisting of liquid ingredients, solid ingredients, powered ingredients and combinations thereof.

7. The system of claim 5, wherein the first feeding line and the second feeding line are selected from a group consisting of pumps, conveyors, auger feeders and combinations thereof.

8. The system of claim 5, wherein the first chemical composition sensor and the second chemical composition sensor is a transducer selected from the group consisting of electrochemical transducer, optical transducer, mass transducer, thermal transducer, acoustic transducer and combinations thereof.

9. The system of claim 8, wherein the optical transducer is selected from the group consisting of optical transmission transducer, optical reflectance transducer, dispersion transducer, fluorescence transducer and combinations thereof.
